(19) United States
(12) Patent Application Publication
(10) Pub. No.: US 2011/0230961 A1
(43) Pub. Date: Sep. 22, 2011

(54) DYNAMICALLY ADJUSTABLE ANNULOPLASTY RING AND PAPILLARY MUSCLE REPOSITIONING SUTURE

(75) Inventors: Frank Langer, Zweibrucken (DE); Hans-Joachim Schäfers, Homburg (DE); Samuel M. Shaolian, Newport Beach, CA (US); Paul A. Molloy, San Clemente, CA (US); Ross Tsukashima, San Diego, CA (US)

(73) Assignee: MICARDIA CORPORATION, Irvine, CA (US)

(21) Appl. No.: 12/985,066

(22) Filed: Jan. 5, 2011

Related U.S. Application Data
(60) Provisional application No. 61/292,390, filed on Jan. 5, 2010.

(51) Int. Cl. A61F 2/24

(52) U.S. Cl. 623/2.27

(57) ABSTRACT

A system for treating a cardiac valve includes an adjustable annuloplasty ring configured to attach to or near a cardiac valve annulus. The system also includes a suture comprising a first end coupled to the annuloplasty ring. A second end of the suture is configured to be anchored to a papillary muscle. Selectively adjusting the annuloplasty ring adjusts a tension of the suture to reposition the papillary muscle.

### RELATED APPLICATION

This application claims the benefit under 35 U.S.C. §119(e) of U.S. Provisional Application No. 61/292,390, filed Jan. 5, 2010, which is hereby incorporated by reference herein in its entirety.

### TECHNICAL FIELD

This disclosure relates generally to medical devices and methods for repairing a defective heart valve. More specifically, this disclosure relates to medical devices and methods for treating heart valve regurgitation.

### BRIEF DESCRIPTION OF THE DRAWINGS

Non-limiting and non-exhaustive embodiments of the disclosure are described, including various embodiments of the disclosure with reference to the figures, in which:

FIG. 1 is a first cross-sectional view of a typical four-chambered heart in which the position of all four heart valves can be identified.

FIG. 2 is a second cross-sectional view of a typical four-chambered heart, wherein the mitral valve and tricuspid valve are viewable.

FIG. 3A is a perspective view of a normal mitral valve having proper coaptation of the anterior and posterior leaflets.

FIG. 3B is a cross-sectional view of the mitral valve of FIG. 3A.

FIG. 4 is a perspective view of an annuloplasty ring.

FIG. 5 is a schematic representation of the transventricular suture technique.

FIG. 6 schematically illustrates a system including a dynamically adjustable annuloplasty ring and papillary muscle repositioning suture, according to one embodiment of the present disclosure.

FIGS. 7, 8A, 8B, 9, 10, 11, 12, and 13 schematically illustrate other embodiments of a dynamically adjustable annuloplasty ring and suture disclosed herein.

FIGS. 14A and 14B schematically illustrate a papillary muscle repositioning suture coupled to a dynamically adjustable annuloplasty ring including a motor, according to one embodiment of the present disclosure.

FIGS. 15A and 15B schematically illustrate another embodiment of a papillary muscle repositioning suture coupled to a dynamically adjustable annuloplasty ring including a motor.

FIG. 16 schematically illustrates still another embodiment of a papillary muscle repositioning suture coupled to a dynamically adjustable annuloplasty ring including a motor.

FIG. 17A is a block diagram of a system for adjusting the size of a heart valve according to one embodiment that includes an annuloplasty ring and an external magnetic driver or adjustment device.

FIG. 17B is an enlarged, cross-sectional view of the annuloplasty ring and the external magnetic adjustment device shown in FIG. 17A, according to one embodiment.

FIGS. 18A and 18B schematically illustrate a magnet that is usable in the annuloplasty ring shown in FIG. 17A, according to one embodiment.

FIGS. 19A and 19B schematically illustrate an end view of the magnet of the external magnetic adjustment device placed in parallel with the magnet of the annuloplasty ring, according to certain embodiments.

FIG. 20 is a schematic diagram of an external magnetic adjustment device including two magnets arranged outside of a patient's body, according to one embodiment.

### DETAILED DESCRIPTION

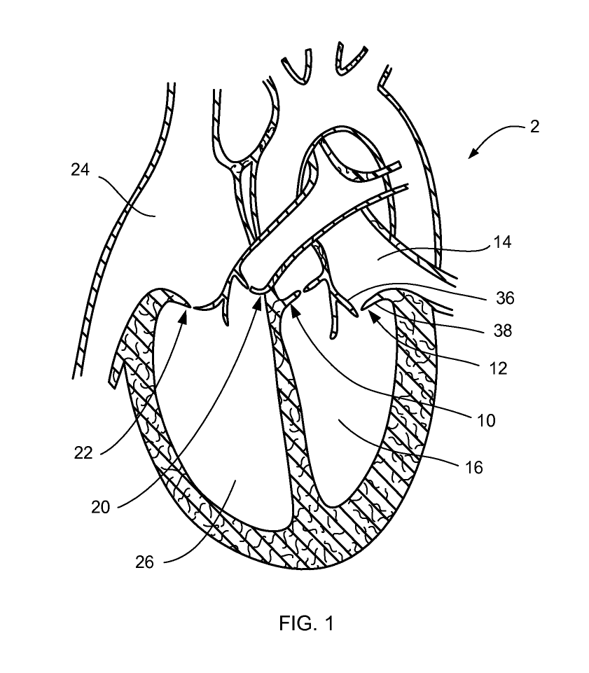

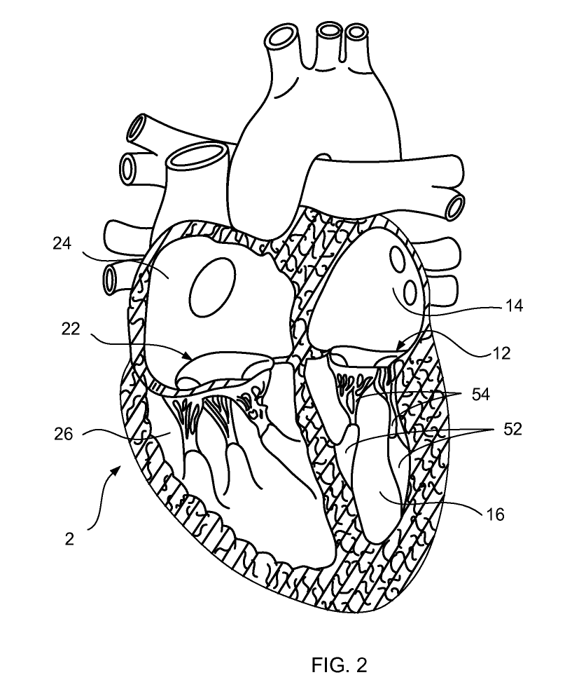

FIG. 1 is a first cross-sectional view of a human heart 2 in which all four heart valves can be seen. FIG. 2 is a second cross-sectional view of a human heart 2, wherein the mitral valve and tricuspid valve are depicted. The human heart 2 is four-chambered and has four valves that control the direction of blood flow in the circulatory system. The aortic valve 10 and mitral valve 12 are part of the “left” heart and control the flow of oxygen-rich blood from the lungs to the peripheral circulation, while the pulmonary valve 20 and tricuspid valve 22 are part of the “right” heart and control the flow of oxygen-depleted blood, returning from the body, to the lungs. The aortic valve 10 and pulmonary valve 20 lie between a ventricle 16, 26 (pumping chamber) and a major artery, preventing blood from leaking back into the ventricle 16, 26 after being ejected into the circulatory system. The mitral valve 12 and tricuspid valve 22 lie between an atrium 14, 24 (receiving chamber) and a ventricle, 16, 26 preventing blood from flowing back into the atrium 14, 24 during ventricular contraction.

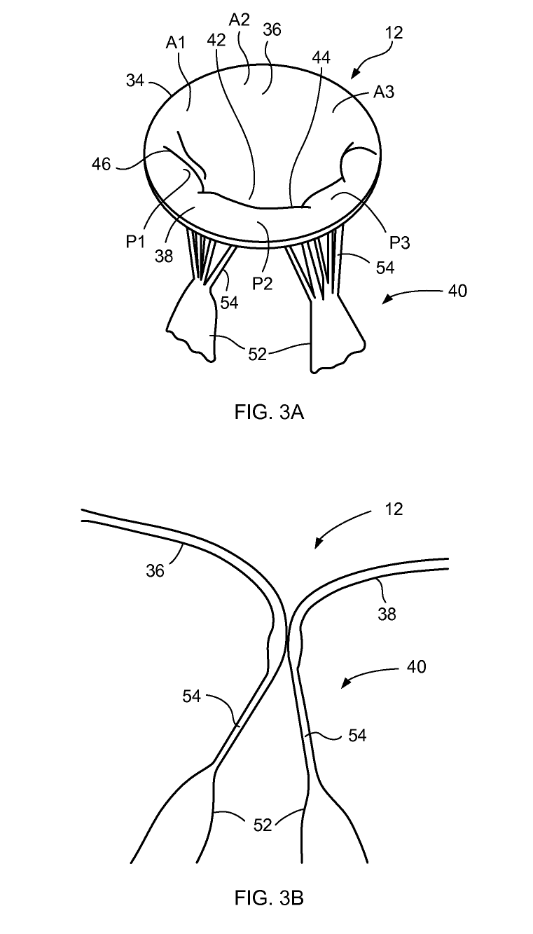

A normal mitral valve 12, an example of which is more closely illustrated in FIGS. 3A and 3B, can be divided into three parts, an annulus 34, a pair of leaflets 36, 38 and a sub-valvular apparatus 40. The annulus 34 is a dense ring of fibrous tissue which lies at the juncture between the left atrium 14 and the left ventricle 16. The annulus 34 is normally elliptical, or “kidney-shaped,” with a vertical (anteroposterior) diameter approximately three-fourths of the transverse diameter. The larger elliptical anterior leaflet 36 and the smaller, crescent-shaped posterior leaflet 38 attach to the annulus 34. Approximately three-fifths of the circumference of the annulus 34 is attached to the posterior leaflet 38 and two-fifths of the annular circumference is attached to the anterior leaflet 36. The edge of each leaflet not attached to the annulus 34 is known as the free margin 42.

When the valve is closed, the free margins 42 of the two leaflets come together within the valve orifice forming an arc known as the line of coaptation 44. The points on the annulus where the anterior and posterior leaflets meet, are known as commissures 46. The posterior leaflet 38 is usually separated into three distinct scallops by small clefts. The posterior scallops are referred to (from left to right) as P1 (anterior scallop), P2 (middle scallop) and P3 (posterior scallop). The corresponding segments of the anterior leaflet directly opposite P1, P2 and P3 are referred to as A1 (anterior segment), A2 (middle segment) and A3 (posterior segment).

The sub-valvular apparatus 40 of the mitral valve 12 includes two thumb-like muscular projections from the inner wall of the left ventricle 16 (as seen in FIG. 2) known as papillary muscles 52 and numerous chordae tendinae 54 (also referred to simply as chordae). The chordae 54 are thin fibrous bundles that emanate from the tips of the papillary muscles 52 and attach to the free margin 42 or undersurface of the valve leaflets 36, 38 in a parachute-like configuration. The chordae 54 are classified according to their site of attachment between the free margin 42 and the base of the leaflets 36, 38. Marginal, or primary, chordae are attached at the free margin 42 of the leaflets 36, 38 and function to limit leaflet prolapse. Intermediate, or secondary, chordae are attached to the underside of the leaflets 36, 38 at points between the free margin 42 and the base of the leaflets 36, 38. Basal, or tertiary, chordae are attached to the base of the leaflets 36, 38.

Normally, the mitral valve 12 opens when the left ventricle 16 relaxes (diastole) allowing blood from the left atrium 14 to fill the left ventricle 16. When the left ventricle 16 contracts (systole), the increase in pressure within the ventricle 16 causes the mitral valve 12 to close, preventing blood leakage back into the left atrium 14, and ensuring that substantially all of the blood leaving the left ventricle (i.e., the stroke volume) is ejected through the aortic valve 10 into the aorta and to the peripheral circulation of the body. Proper function of the mitral valve 12 is dependent on a complex interplay between the annulus 34, the leaflets 36, 38 and the sub-valvular apparatus 40.

Various disease processes can impair the proper functioning of one or more of the heart valves 10, 12, 20, 22. These include degenerative processes (e.g., Barlow's Disease, fibroelastic deficiency), inflammatory processes (e.g., rheumatic heart disease), and infectious processes (e.g., endocarditis). In addition, damage from heart attack, or other heart diseases (e.g., cardiomyopathy), can distort valve geometry and lead to diminished functionality.

Heart valves can malfunction in one of two ways. Valve stenosis describes the situation where the valve does not open completely, resulting in an obstruction to blood flow. Valve regurgitation describes the situation where the valve does not close completely, resulting in leakage back into a heart chamber against the normal direction of flow (e.g., leakage from a ventricle back to an atrium, or from the circulation back to a ventricle). The present disclosure is described primarily in relation to valve regurgitation, and in particular regurgitation occurring in the mitral valve 12. An ordinarily skilled artisan will appreciate, however, that the concepts disclosed also may be applicable to valve regurgitation in any of the four heart valves 10, 12, 20, 22, and there may be instances where the concepts and ideas disclosed may be applicable in relation to valve stenosis.

Referring specifically to the mitral valve 12, regurgitation results in backflow of blood from the left ventricle 16 to the left atrium 14 during systole, a condition known as mitral regurgitation. Since a portion of cardiac output is wasted when blood flows back into the left atrium 14, the heart 2 must work harder in order to pump the volume of blood needed to maintain proper perfusion of tissues in the body. Over time, this increased workload leads to myocardial remodeling in the form of left ventricular dilation, or hypertrophy. Mitral valve regurgitation can also lead to increased pressures in the left atrium, which may result in a back up of blood in the venous circulation, and fluid in the tissues of the body, a condition known as congestive heart failure.

Mitral valve dysfunction leading to mitral regurgitation can be classified into three types based on the motion of the leaflets 36, 38 (commonly known as “Carpentier's Functional Classification”). Type I dysfunction generally does not affect normal leaflet motion. Mitral regurgitation in patients exhibiting Type I dysfunction can be due to perforation of the leaflet 36, 38 (usually from infection) or, much more commonly, can result from distortion or dilation of the annulus 34. Annular dilation/distortion causes separation of the free margins 42 of the two leaflets 36, 38, producing a gap. This gap prevents the leaflets 36, 38 from fully coapting, in turn allowing blood to leak back into the left atrium 14 during systolic contraction.

Type II dysfunction results from leaflet prolapse. This occurs when a portion of the free margin 42 of one, or both, leaflets 36, 38 is not properly supported by the sub-valvular apparatus 40. During systolic contraction, the free margins 42 of the involved portions of the leaflets 36, 38 prolapse above the plane of the annulus 34 and into the left atrium 14. This prevents leaflet coaptation and again allows blood to regurgitate into the left atrium 14 between the leaflets 36, 38. The most common causes of Type II dysfunction include chordal or papillary muscle elongation, or rupture, due to degenerative changes (such as myxomatous pathology or Barlow's Disease and fibroelastic deficiency), or prior myocardial infarction.

Type III dysfunction results from restricted leaflet motion. Here, the free margins 42 of portions of one or both leaflets 36, 38 are pulled below the plane of the annulus 34 into the left ventricle 16. Leaflet motion that is restricted during both systole and diastole is termed a Type III A dysfunction. The restricted leaflet motion can be related to valvular or sub-valvular pathology including leaflet thickening or retraction, chordal thickening, shortening or fusion and commissural fission, any or all of which can be associated with some degree of stenosis or fibrosis. Leaflet motion that is restricted during systole only is termed a Type III B dysfunction. Specifically, the leaflets 36, 38 are prevented from rising up to the plane of the annulus 34 and coapting during systolic contraction. The resulting leaflet tethering and displacement of the coaptation point toward the ventricle 16 are geometric distortions that are commonly described as “tenting.” This type of dysfunction most commonly occurs when abnormal ventricular geometry or function, usually resulting from prior myocardial infarction (“ischemia”) or severe ventricular dilatation and dysfunction (“cardiomyopathy”), leads to papillary muscle displacement. The otherwise normal leaflets 36, 38 are pulled down into the ventricle 16 and away from each other, preventing proper coaptation.

Treatment options for malfunctioning heart valves can include valve repair, preserving the patient's natural valve, or replacement with a mechanical, or biologically-derived, substitute valve. Since there are well known disadvantages associated with the use of valve prostheses, including increased clotting risk, and limited durability of the replacement valve, repair is usually preferable, when possible, to replacement. In many cases, however, valve repair is usually more technically demanding than replacement.

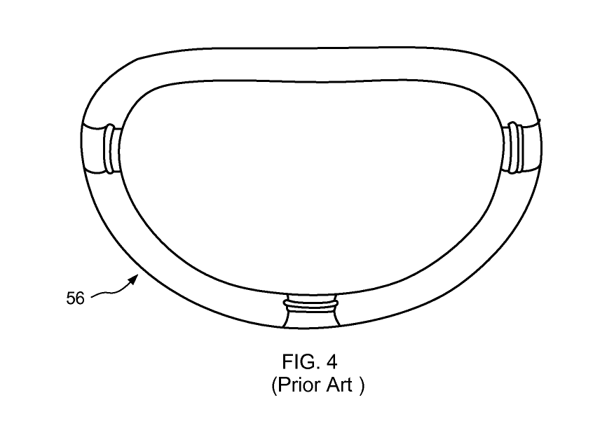

Ring annuloplasty is a standard surgical repair technique for ischemic mitral regurgitation (IMR). A ridged ring, like the annuloplasty ring 56 shown in FIG. 4 is sewn around the mitral valve 12 with an aim to reduce the valve annulus 34. Cinching the tissue around the ring 56 can restore the valve annulus 34 to its approximate original size and operating efficiency. The proper degree of cinching, however, is difficult to determine and achieve during open heart surgery. This is because the patient is under general anesthesia, in a prone position, with the chest wide open, and a large incision in the heart. These factors and others affect the ability to test the modified annulus 34 for its therapeutic affect upon mitral valve leaflet coaptation. Even if the cinching is done well, the tissue may continue to change over the patient's lifetime such that the heart condition returns. A dynamically adjustable ring that can be adjusted after surgery enables proper cinching to occur after surgery.

Ring annuloplasty has proven to be effective in many cases, but residual or recurrent mitral regurgitation (MR) after ring annuloplasty is seen in up to 30% of patients. Annular reduction may in some instances correct both annular and sub-valvular geometry in IMR, but annular reduction using ring annuloplasty primarily addresses only the annular dilation dysfunction that causes IMR. Altered sub-valvular geometry simply may not be sufficiently addressed by undersized annular reduction, particularly in cases where tenting is severe (e.g., tenting height exceeding 10 mm). More specifically, undersized ring annuloplasty may fail to correct papillary muscle displacement sufficiently to eliminate tethering of the leaflet(s) due to the displacement. Papillary muscle displacement can cause increased tension on the chordae tendinae. The chordae tendinae play an important role in correct valve coaptation by connecting the leaflets of the valve to the papillary muscle. The increased tension on the chordae tendinae results in leaflet tethering. When leaflet tethering persists, residual or recurrent mitral regurgitation can result.

One method for addressing altered sub-valvular geometry is the transventricular suture technique, which involves surgical repositioning of the displaced papillary muscle using a sub-valvular transventricular suture. FIG. 5 is a schematic representation of the transventricular suture technique in a human heart 2. A suture 58 is anchored (shown as anchor 59) in the head of the posterior papillary muscle 52 at a point near the annulus of the mitral valve 12. The suture 58, with a properly adjusted tension, can function similar to a cable in a suspension bridge to raise or otherwise reposition the papillary muscle 52 and effectively unload the chordae tendinae 54. Elevating the papillary muscle 52 to which the chordae 54 are attached relieves tension on associated leaflets. Reducing tension on the leaflets may allow the leaflets to function properly, thereby reducing or eliminating valve regurgitation.

Achieving the proper degree of tension on the suture 58 is difficult during open heart surgery. This is because the patient is under general anesthesia, in a prone position, with the chest wide open, and a large incision in the heart. These factors and others affect the ability to assess the effect of repositioning of the papillary muscle and tension of the suture 58 and/or the chordae tendinae 54. Even if the tension of the suture 58 is properly adjusted, the tissue may continue to change over the patient's lifetime such that the heart condition returns. Thus, according to certain embodiments disclosed herein, a dynamically adjustable suture allows adjustment of the tension after surgery.

Accordingly, the present disclosure contemplates devices and methods providing a combined adjustable annuloplasty ring and adjustable suture attached to the adjustable ring that may be used in heart valve repair. Combining the adjustable ring and adjustable suture may enable sub-valvular repair in conjunction with ring annuloplasty techniques. The ring, after insertion at the annulus of the mitral valve, provides a suitable anchor point for one end of the suture, thereby eliminating one step in anchoring the suture. Moreover, making the suture dynamically adjustable enables dynamic repositioning of a papillary muscle to thereby achieve proper tension of the suture to appropriately unload (i.e., decrease tension on) the chordae tendinae.

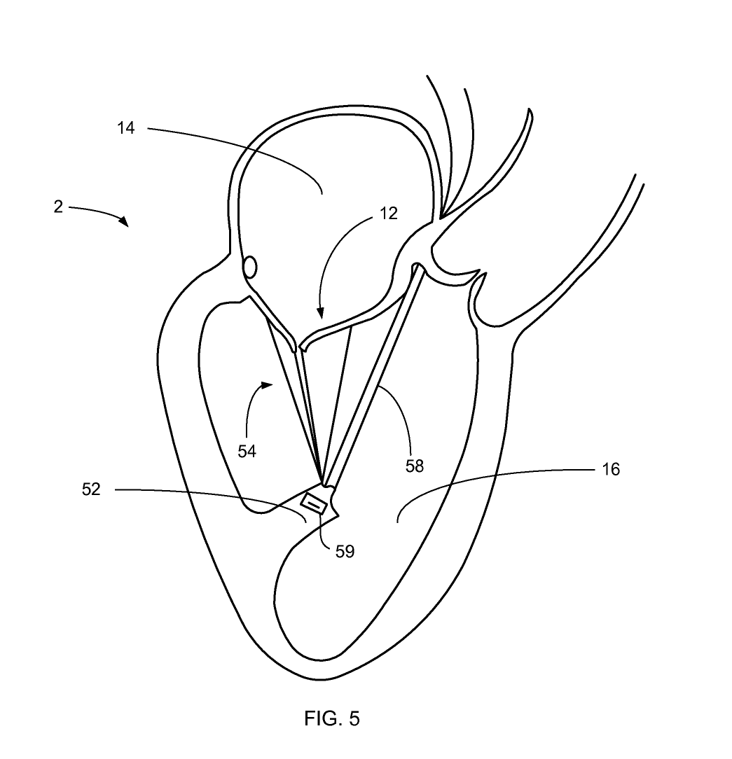

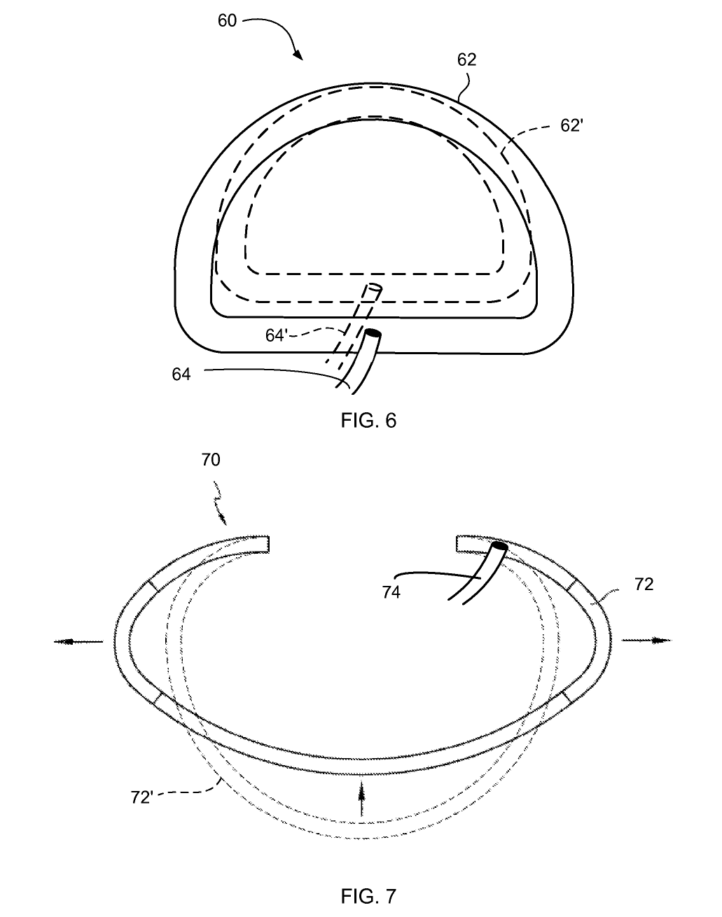

FIG. 6 schematically illustrates a system 60 for treating a cardiac valve according to one embodiment of the present disclosure. The system 60 includes a suture 64 coupled to a dynamically adjustable annuloplasty ring 62. In the illustrated embodiment, the suture 64 is attached to the annuloplasty ring 62 at the midpoint of the A2 region (see FIG. 3A) of the ring 62 (corresponding to the mid-septal fibrous annulus or annular saddle horn), to allow for a desired force vector on the papillary muscle. The annuloplasty ring 62 may be formed of a shape memory plastic, shape memory alloy, or other material configured to shrink or otherwise change shape in response to a stimulus, such as heat, a magnetic field, or an electrical impulse. More complete details of example embodiments of dynamically adjustable annuloplasty rings can be found in U.S. Patent Application Publication No. 2009/0088838, which is assigned to the assignee of the present disclosure and is hereby incorporated by reference herein for all purposes. In the instant embodiment, the annuloplasty ring 62 may lie in a plane and be configured to change shape within the plane. As the annuloplasty ring 62 changes shape, the annuloplasty ring 62 pulls one end of the suture 64, thereby increasing tension on the suture 64 to displace a papillary muscle (e.g., the papillary muscle 52 shown in FIG. 5) to which the other end (not shown) of the suture 64 is anchored. For illustrative purposes in FIG. 6, the shape of the annuloplasty ring 62′ after a shape change and the adjusted suture 64′ are both illustrated in broken lines.

FIG. 7 schematically illustrates another embodiment of a device 70 for treating heart valves, according to the present disclosure. Again, the device 70 includes a suture 74 coupled to a dynamically adjustable ring-like component 72 (referred to herein as a “ring”). In the illustrated embodiment, the ring has a “C” shape, and the suture 74 is attached near an end of the “C” in approximately the A3 region (see FIG. 3A) of the ring 72 (corresponding to the right fibrous trigone). In another embodiment, the suture 74 may be attached to the other end of the “C” in approximately the A1 region (see FIG. 3A) of the ring 72. In still another embodiment, multiple sutures may be attached to the ring 72, for example at both the A1 and A3 regions of the ring. The ring 72 may be formed of a shape memory plastic, a shape memory alloy, or other material configured to shrink or otherwise change shape in response to a stimulus. The shape and/or size of the ring 72 can dynamically adjust in response to a stimulus. Similar to the embodiment 60 of FIG. 6, the ring 72 may lie in a plane and dynamically change shape within the plane. In FIG. 7 the shape of the ring 72 after dynamic adjustment is shown, while the original shape of the ring 72′ is shown in broken lines. Arrows indicate the direction of the change in shape. For clarity, the repositioned suture is not shown in FIG. 7. However, an artisan will recognize from the disclosure herein that dynamically reshaping the implanted ring 72 in the directions indicated by the arrows in FIG. 7 would result in adjusting the tension of the suture 74 and repositioning the papillary muscle (e.g., the papillary muscle 52 shown in FIG. 5).

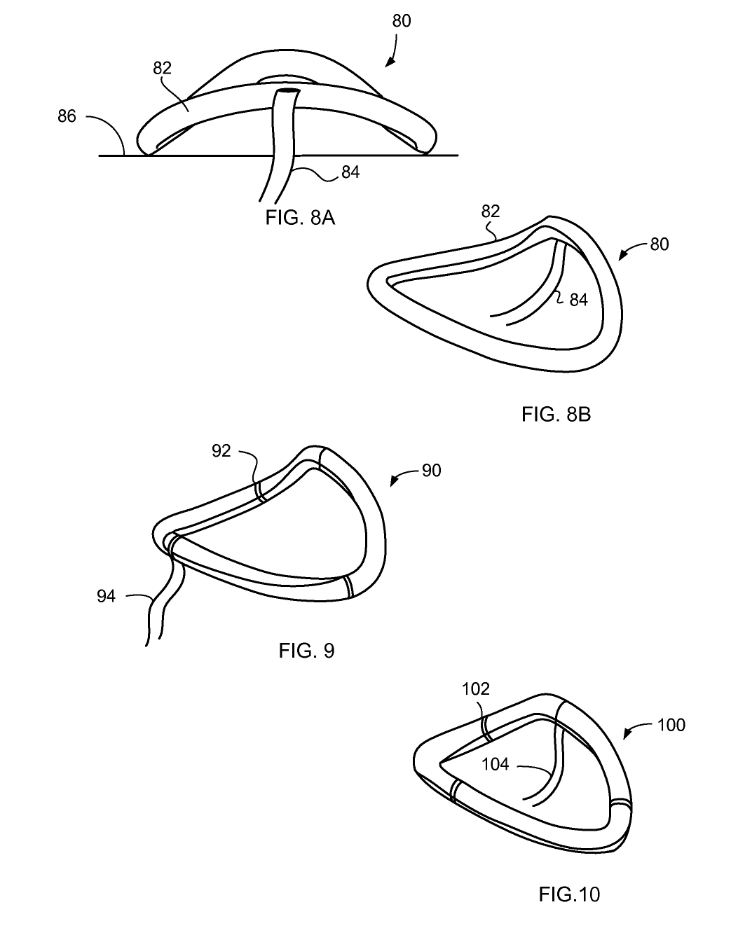

FIGS. 8A and 8B schematically illustrate another embodiment of a device 80 according to the present disclosure. FIG. 8A is a side view and FIG. 8B is a perspective view of the device 80 and FIG. 8B is a perspective view of the device 80, which includes a suture 84 coupled to a dynamically adjustable ring 82. The shape and/or size of the ring 82 can dynamically adjust in response to a stimulus. The ring 82 may initially lie in a plane 86. FIGS. 8A and 8B depict how the ring can change shape and break the plane, i.e., a three-dimensional shape change. A portion of the ring 82 shifts away from (e.g., above or below) the plane 86. The suture 84 may be coupled to the ring 82 at the portion of the ring 82 that shifts away from the plane 86. Thus, as the ring 82 changes shape in response to a stimulus, the tension of the suture 84 increases.

FIG. 9 is still another embodiment of a device 90 including a dynamically adjustable annuloplasty ring 92 and a suture 94, according to the present disclosure. The suture 94 is attached to the ring 92 at a different location than that of previously described embodiments. The ring 92 changes shape by shifting out of, or away from, a plane of the initial configuration of the ring 92.

FIG. 10 is still another embodiment of a device 100 including a dynamically adjustable annuloplasty ring 102 and a suture 104, according to the present disclosure. The ring 102 has a different shape than that of the previously described and illustrated embodiments. The ring 102 changes shape by shifting out of, or away from, a plane of the initial configuration of the ring 102. The suture 104 is coupled to the ring 102.

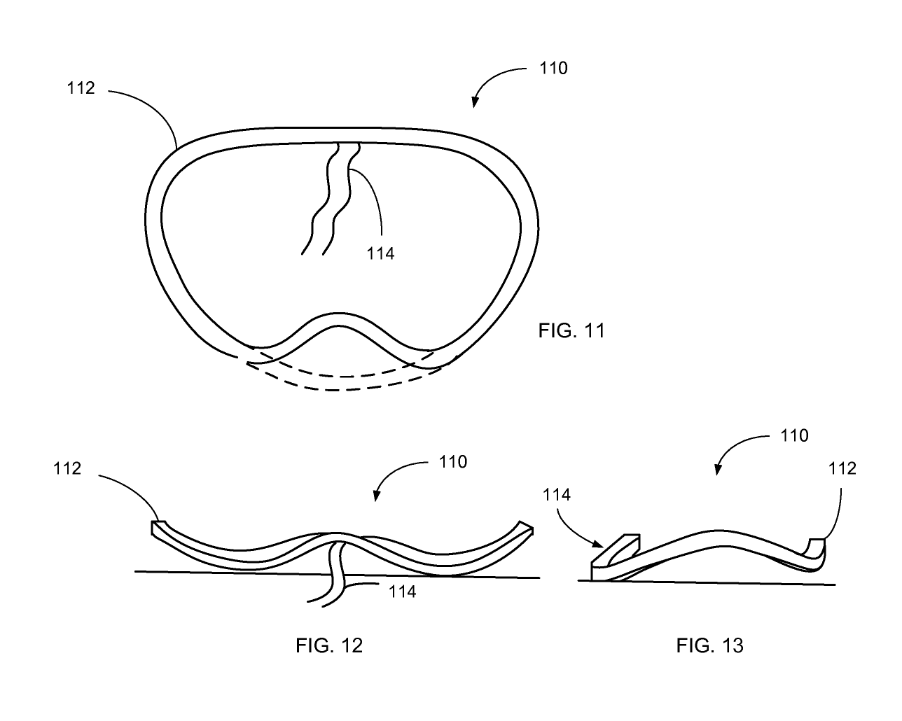

FIGS. 11, 12, and 13 are still other embodiments of a device 110 including a dynamically adjustable annuloplasty ring 112 and a suture 114. FIG. 11 is a top view of the device 110. FIG. 12 is a front side view of the device 110 of FIG. 11. FIG. 13 is a lateral side view of the device 110 of FIGS. 11 and 12. The device 110 includes a ring 112 formed of shape memory plastic, shape memory alloy, or other material configured to shrink or otherwise change shape in response to a stimulus. As illustrated, the ring 112 changes shape by portions of the ring 112 shifting both within the plane and out of the plane. The shape change can increase tension on a suture 114. As shown in FIGS. 11 and 12, the suture 114 may be attached at various locations of the ring 112, depending on the particular application and/or desired repositioning of the papillary muscle.

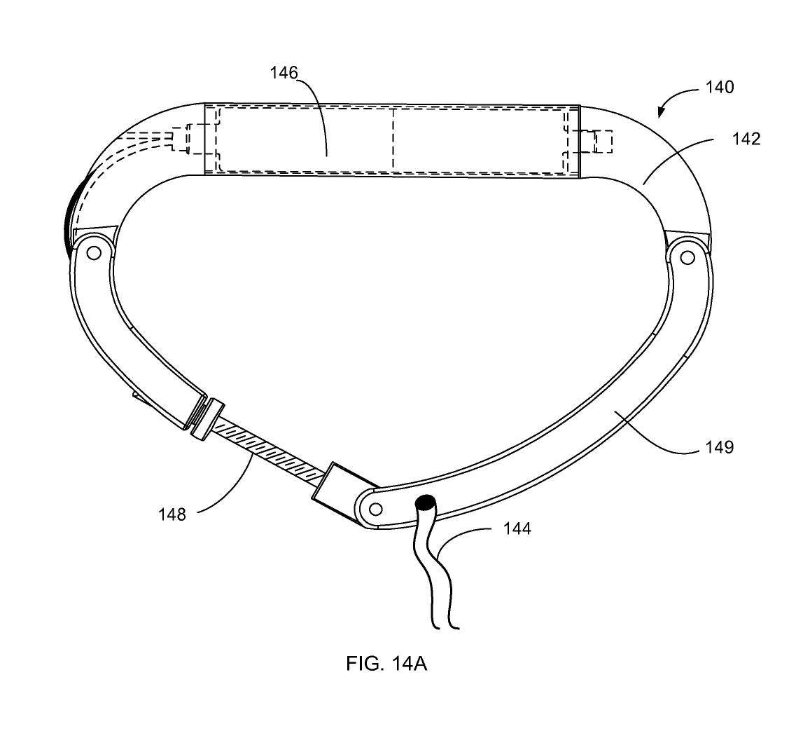

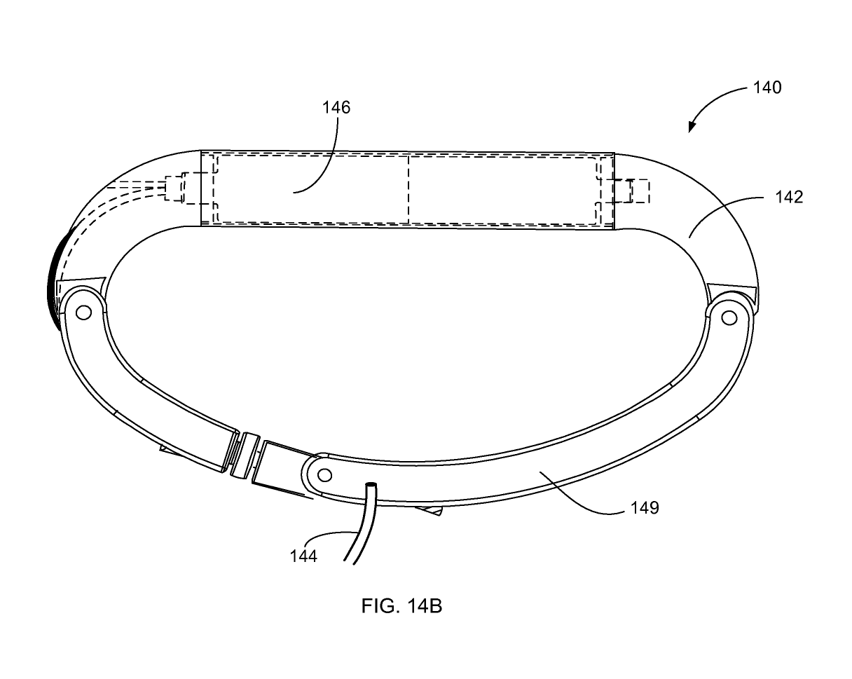

FIGS. 14A and 14B schematically illustrate a device 140, according to another embodiment. The device 140 includes a suture 144 coupled to a dynamically adjustable annuloplasty ring 142. The annuloplasty ring 142 includes a motor 146 configured to drive a rod 148 (including a flexible rod or cable) that adjusts the size of the annuloplasty ring 142. The motor 146 may be magnetically driven. Additional details of example embodiments of a dynamically adjustable annuloplasty ring with a magnetically driven motor are disclosed in U.S. Patent Application Publication No. 2009/0248148, which is hereby incorporated by reference herein for all purposes. In other embodiments the motor 146 may be electrically driven and a battery may power the motor 146. The suture 144 may couple to an adjustable portion (e.g., an adjustable arm 149) of the ring 142, as shown. When the shape of the ring 142 is adjusted, the tension on the suture 144 is also adjusted.

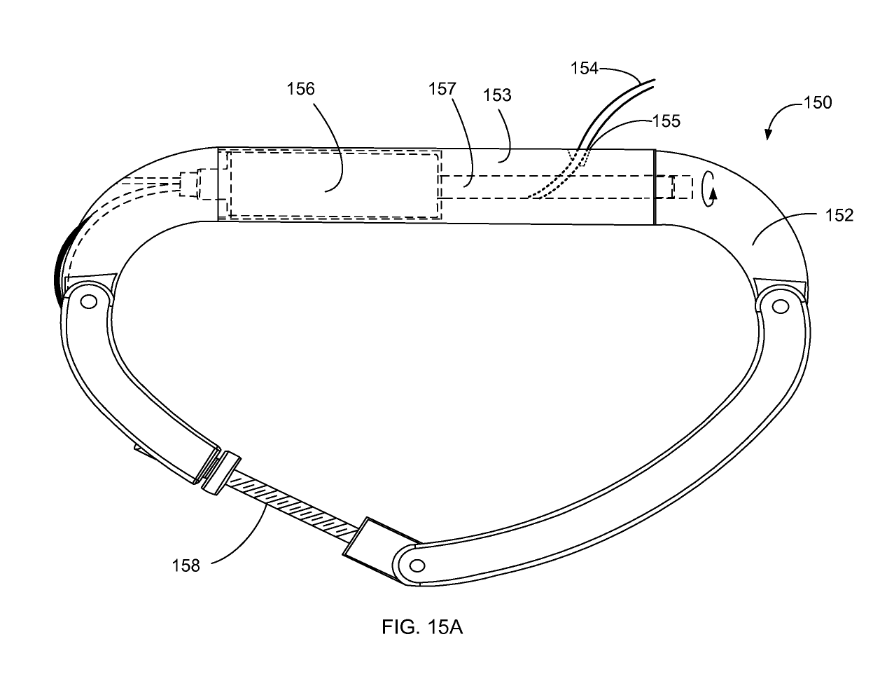

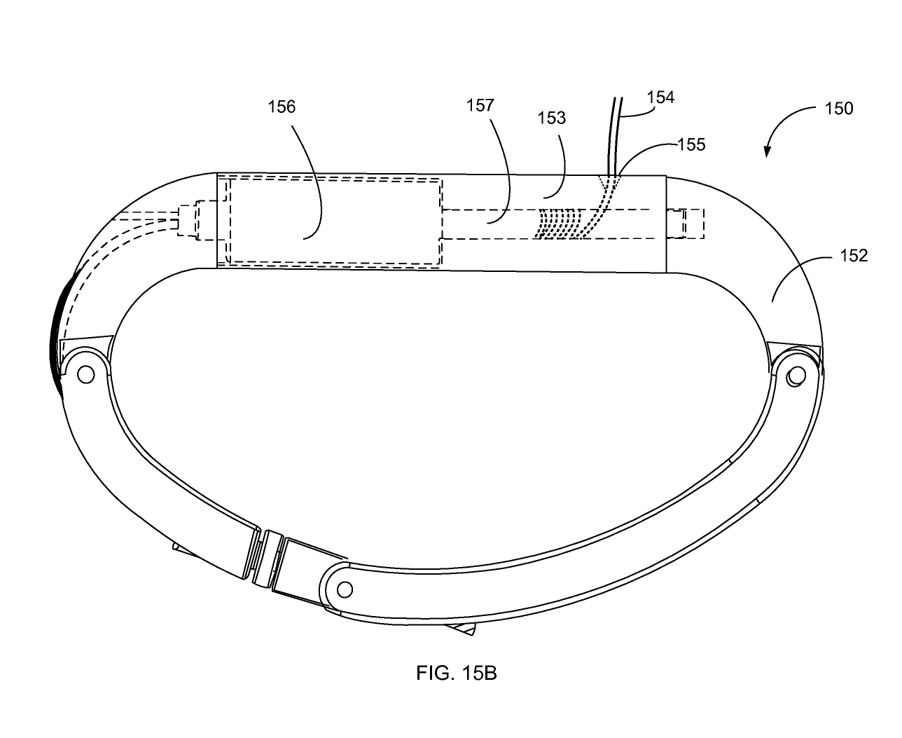

FIGS. 15A and 15B schematically illustrate a device 150, according to another embodiment. The device 150 includes a dynamically adjustable annuloplasty ring 152 having a motor 156 and a suture 154 coupled to the motor 156. The motor 156 is configured to drive a rod 158 (which may include a rigid rod, a flexible rod, and/or a cable) that is in turn configured to adjust the size of the annuloplasty ring 152. As shown, the motor 156 may include, or may be coupled to, a spool 157. A funnel 155 allows the suture to enter the hollow body 153 of the ring 152, which houses the motor 156. The funnel 155 may comprise PTFE or another suitable material that may be implanted within a patient. Inside the hollow body 153, the suture 154 couples to the spool 157. As the motor 156 rotates to drive the rod 158 and adjust the size of the ring 152, the motor 16 also rotates the spool 157. The spool 157 selectively rotates in either direction, winding and unwinding the suture 154 as shown in FIG. 15B, thereby adjusting the tension of the suture 154. The motor 156 may be internally sealed within the hollow housing to prevent degradation and dysfunction of the motor 156, which may result from fluid and/or debris accessing the motor 156.

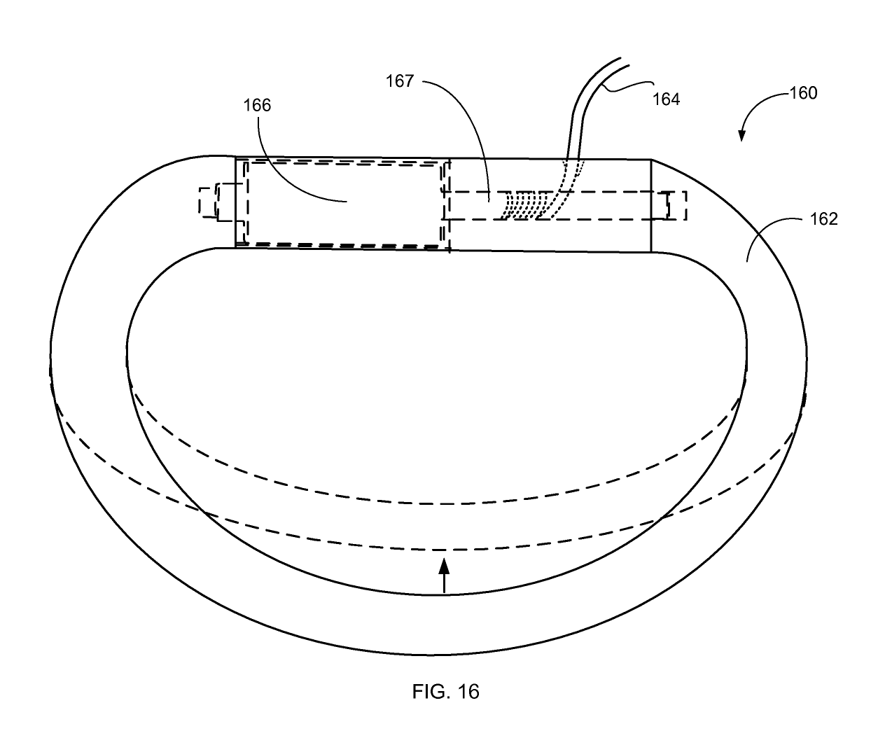

FIG. 16 schematically illustrates a device 160, according to another embodiment. The device 160 includes a dynamically adjustable annuloplasty ring 162 having a motor 166 and a suture 164. The annuloplasty ring 162 may be formed of a shape memory material configured to shrink or otherwise change shape in response to a stimulus. The shape of the ring 162 after changing is shown in broken lines. The ring 162 also houses a motor 166 configured to wind up the suture 164 and thereby adjust the tension of the suture 164. Accordingly, the ring 162 and the suture 164 are separately adjustable. The size and shape of the ring 162 can be adjusted by providing a stimulus (e.g., heat, magnetic field, electrical impulse), and the tension of the suture 164 can be adjusted by the motor 166. The motor 166 is coupled to a spool 167 to selectively wind and unwind the suture 164. In certain embodiments, activating the shape memory material also adjusts the tension of the suture 164 such that the suture 164 may be separately adjusted by activating the shape memory material, rotating the spool 167 using the motor 166, or both.

In still another embodiment, the spool coupled to the motor may be replaced by a rod that is pulled or pushed laterally relative to the motor. As the motor turns, the rod is pulled into the motor or pushed out and away from the motor. For example, the rod may be threaded and coupled to complementary threads on the motor. As the motor rotates, the rod may not rotate, such that the threads of the motor cause lateral displacement of the threads of the rod. A suture may be coupled to the rod and the tension of the suture may be adjusted as the rod moves laterally relative to the motor.

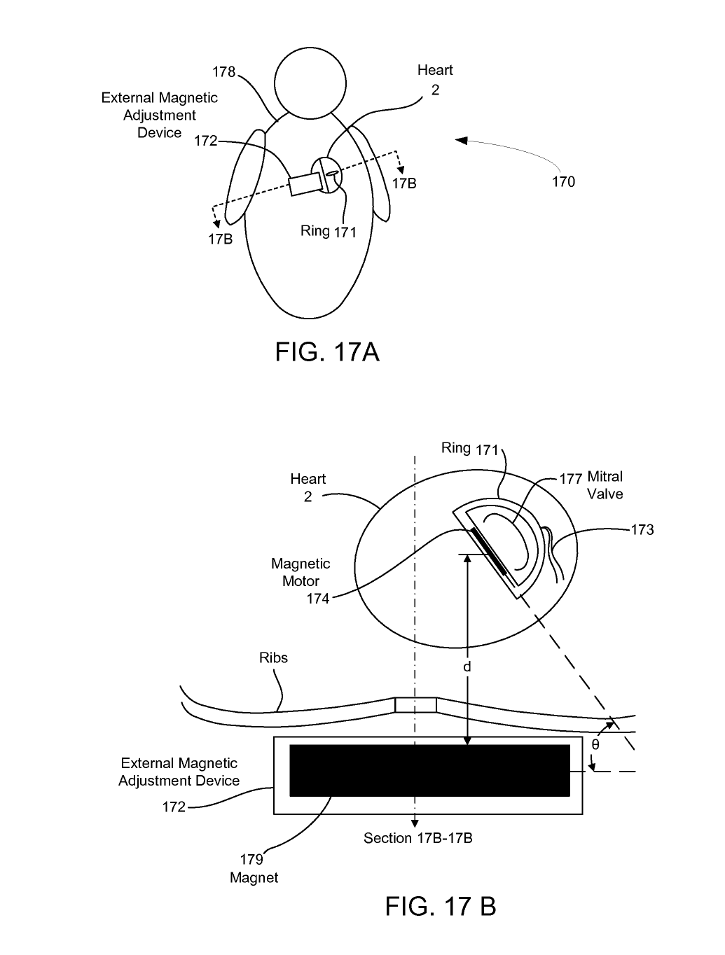

FIG. 17A is a block diagram of a system 170 for treating heart valve regurgitation. The system includes a dynamically adjustable annuloplasty ring 171 and an external magnetic driver or adjustment device 172. For illustrative purposes, FIG. 17B is an enlarged, cross-sectional view of the annuloplasty ring 171 and the external magnetic adjustment device 172 shown in FIG. 1A. The adjustable annuloplasty ring 171 may include a suture 173 coupled to a dynamically adjustable portion of the annuloplasty ring, and a magnetic motor 174 to adjust the dynamically adjustable portion of the annuloplasty ring 171. As disclosed herein, the suture 173 is used to reposition the papillary muscle. The annuloplasty ring 171 may be implanted in a heart 2 of a patient 178 in the same manner as current rigid annuloplasty rings. The annuloplasty ring 171 in this example is “D” shaped and may be attached, for example, to the mitral valve 177. However, an artisan will recognize from the disclosure herein that other shapes (e.g., circular or “C” shaped rings) may also be used and that other openings (e.g., for the tricuspid valve) may be treated.

The magnetic motor 174 of the adjustable annuloplasty ring 171 may include a permanent magnet that may be rotated remotely by one or more magnets 179 in the external magnetic adjustment device 172. Rotating the one or more magnets 179 in the external magnetic adjustment device 172 in one direction causes the annuloplasty ring 171 to close while turning the one or more magnets 179 in the opposite direction causes the annuloplasty ring 171 to open. The external magnetic adjustment device 172 shown in FIGS. 17A and 17B may include an external hand piece that controls the annuloplasty ring 171 from outside of the patient's body at a distance d from the annuloplasty ring 171. However, other adjustment devices (including percutaneous adjustment devices) may also be used.

FIGS. 18A and 18B schematically illustrate a magnet 180 that is usable in the motor 174 of the annuloplasty ring 171 shown in FIG. 17A, according to one embodiment. A similarly configured magnet may also be used for a magnet 179 in the external magnetic adjustment device 172. The magnet 180 in this example embodiment is cylindrical and has magnetic poles (e.g., north “N” and south “S”) divided along a plane 181 that runs the length of the cylinder. A rotating magnetic field causes the magnet 180 to rotate around an axis 182 of the cylinder that passes through the respective centers of the cylinder's bases (the “cylindrical axis”).

For example, FIGS. 19A and 19B schematically illustrate an end view of the magnet 179 of the external magnetic adjustment device 172 placed in parallel with the magnet 180 of the annuloplasty ring 171, according to certain embodiments. For illustrative purposes, FIG. 19A illustrates the magnets 179, 180 aligned for maximum (peak) torque transmission and FIG. 19B illustrates the south pole of the magnet 179 of the external magnetic adjustment device 172 aligned with the north pole of the magnet 180 of the annuloplasty ring 171. Regardless of a current or initial alignment of the magnets 179, 180, the magnetic fields of the respective magnets 179, 180 interact with each other such that mechanically rotating the magnet 179 (e.g., using a stepper motor) in the external magnetic adjustment device 172 causes the magnet 180 in the motor 174 of the annuloplasty ring 171 to rotate. For example, rotating the magnet 179 in a clockwise direction around its cylindrical axis causes the magnet 180 to rotate in a counterclockwise direction around its cylindrical axis. Similarly, rotating the magnet 179 in a counterclockwise direction around its cylindrical axis causes the magnet 180 to rotate in a clockwise direction around its cylindrical axis.

The magnet 179 in the external magnetic adjustment device 172 provides accurate one-to-one control of the magnet 180 in the annuloplasty ring 171, assuming sufficient magnetic interaction between the magnets 179, 180. In other words, one complete rotation of the magnet 179 in the external magnetic adjustment device 172 will cause one complete rotation of the magnet 180 in the annuloplasty ring 171. If the relationship between the number of rotations of the magnet 180 and the size of the ring is linear, the size of the annuloplasty ring 171 may be determined directly from the number of revolutions since the ring was at its last known size. If, however, the relationship between the number of revolutions and ring size is not linear, a look-up table based on tested values for a particular ring or type of ring may be used to relate the number of revolutions to the size of the annuloplasty ring 171. Imaging techniques may also be used to determine the ring size after it is implanted in the patient. In addition, or in other embodiments, the annuloplasty ring 171 may include circuitry for counting the number of revolutions or determining its own size, and for communicating this data to a user. For example, the annuloplasty ring 171 may include a radio frequency identification (RFID) tag technology to power and receive data from the annuloplasty ring 171.

While placing the magnets 179, 180 in parallel increases rotational torque on the magnet 180 in the annuloplasty ring 171, the disclosure herein is not so limited. For example, FIG. 17B illustrates that the cylindrical axis of the magnet 179 in the external magnetic adjustment device 172 may be located at an angle θ with respect to the cylindrical axis of the magnet 180 in the annuloplasty ring 171. The rotational torque on the magnet 180 provided by rotating the magnet 179 increases as the angle θ approaches zero degrees, and decreases as the angle θ approaches 90 degrees (assuming both magnets 179, 180 are in the same geometric plane or in parallel planes).

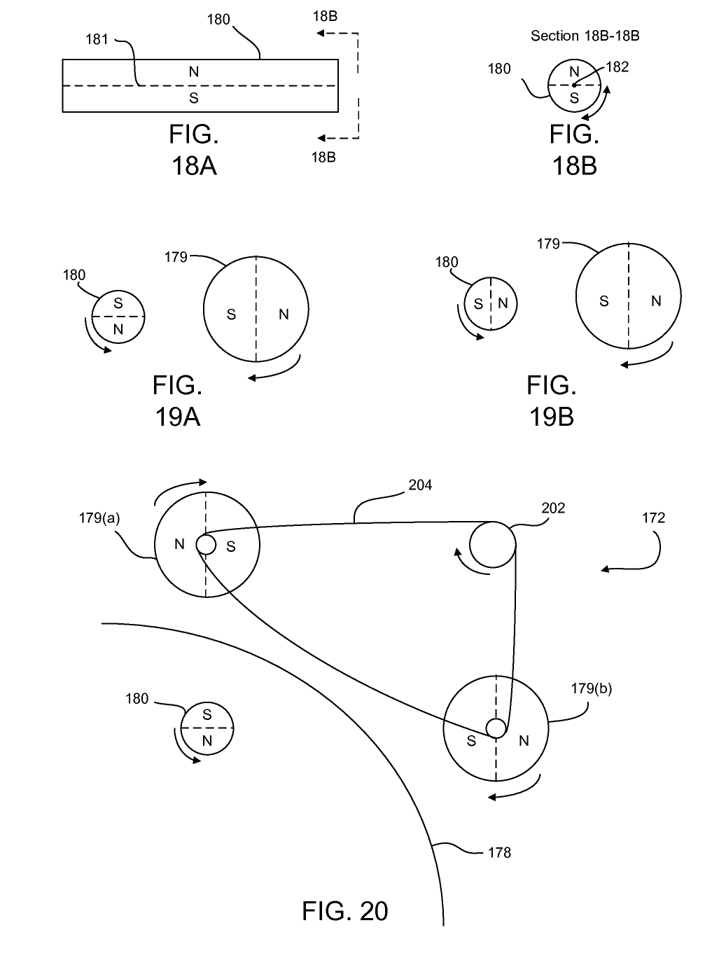

The rotational torque on the magnet 180 in the annuloplasty ring 171 also increases by using magnets 179, 180 with stronger magnetic fields and/or by increasing the number of magnets used in the external magnetic adjustment device 172. For example, FIG. 20 is a schematic diagram of an external magnetic adjustment device 172 including two magnets 179(a), 179(b) arranged outside of a patient's body 178 according to one embodiment. An artisan will recognize from the disclosure herein that the external magnetic adjustment device 172 is not limited to one or two magnets, but may include any number of magnets. The magnets 179(a), 179(b) are oriented and rotated relative to each other such that their magnetic fields add together at the ring magnet 180 to increase rotational torque. A computer controlled motor 202 synchronously rotates the external magnets 179(a), 179(b) through a mechanical linkage 204 to magnetically rotate the internal magnet 180 and adjust the size of the annuloplasty ring 171. One revolution of the motor 202 causes one revolution of the external magnets 179(a), 179(b), which in turn causes one revolution of the ring magnet 180. As discussed above, by counting motor revolutions, the size of the annuloplasty ring 171 may be calculated. In one embodiment, the motor 202 includes a gearbox with a known gear ratio such that multiple motor revolutions may be counted for one magnet revolution.

In another embodiment, a strong electromagnetic field like that used in Magnetic Resonance Imaging (MRI) is used to adjust the annuloplasty ring 171. The magnetic field may be rotated either mechanically or electronically to cause the magnet 180 in the annuloplasty ring 171 to rotate. The patient's body 178 may also be rotated about the axis 182 of the magnet 180 in the presence of a strong magnetic field, like that of an MRI. In such an embodiment, the strong magnetic field will hold the magnet 180 stationary while the annuloplasty ring 171 and patient 178 are rotated around the fixed magnet 180 to cause adjustment. The ring size may be determined by counting the number of revolutions of the magnetic field, or the patient's body, similar to counting revolutions of the permanent magnets 179 discussed above.

In another embodiment, the annuloplasty ring 171 may be adjusted during open heart surgery. For example, after implanting the annuloplasty ring 171 in the heart 2, the heart 2 and pericardium may be closed, and the regurgitation monitored (e.g., using ultrasound color Doppler). Then, a practitioner (e.g., surgeon) may use a handheld device 172 to resize the annuloplasty ring 171 based on the detected regurgitation. Additional regurgitation monitoring and ring adjustment may be performed before completing the surgery.

Various modifications, changes, and variations apparent to those of skill in the art may be made in the arrangement, operation, and details of the methods and systems of the disclosure without departing from the spirit and scope of the disclosure. Thus, it is to be understood that the embodiments described above have been presented by way of example, and not limitation. The scope of the present invention should, therefore, be determined only by the following claims.

1. A system for treating a cardiac valve, the system comprising:
an adjustable annuloplasty ring configured to attach to or near a cardiac valve annulus; and
a suture comprising a first end coupled to the annuloplasty ring, wherein a second end of the suture is configured to be anchored to a papillary muscle,
wherein selectively adjusting the annuloplasty ring adjusts a tension of the suture to reposition the papillary muscle.

2. A system for treating a cardiac valve according to claim 1, wherein the annuloplasty ring comprises a dynamic portion configured to transform in response to a stimulus external to the annuloplasty ring, and wherein the dynamic portion is attached to and adjusts the tension of the suture.

3. A system for treating a cardiac valve according to claim 2, wherein the dynamic portion of the adjustable annuloplasty ring comprises shape-memory material.

4. A system for treating a cardiac valve according to claim 2, wherein the dynamic portion of the adjustable annuloplasty ring is positioned to correspond to the mid-septal fibrous annulus of the heart when the adjustable annuloplasty ring is attached to or near the cardiac valve annulus.

5. A system for treating a cardiac valve according to claim 2, wherein transformation of the dynamic portion changes the size of the annuloplasty ring.

6. A system for treating a cardiac valve according to claim 2, wherein the dynamic portion is configured to transform from a first shape to a second shape.

7. A system for treating a cardiac valve according to claim 6, wherein the first shape lies substantially in a plane and wherein the second shape is a shift of the dynamic portion within the plane.

8. A system for treating a cardiac valve according to claim 6, wherein the first shape lies substantially in a plane and wherein the dynamic portion shifts away from the plane to transform to the second shape.

9. A system for treating a cardiac valve according to claim 2, wherein the annuloplasty ring comprises a motor configured to effect transformation of the dynamic portion.

10. A system for treating a cardiac valve according to claim 9, wherein the dynamic portion of the adjustable annuloplasty ring comprises a hinged portion coupled to a drive rod driven by the motor.

11. A system for treating a cardiac valve according to claim 1, wherein the adjustable annuloplasty ring comprises a motor and operation of the motor adjusts the tension of the suture.

12. A system for treating a cardiac valve according to claim 11, the annuloplasty ring further comprising a spool coupled to the motor and configured to be driven by the motor, wherein the suture is coupled to the spool and configured to wind around the spool or unwind from the spool as the motor turns.

13. A system for treating a cardiac valve according to claim 11, wherein the motor is a magnetic motor comprising an internal magnet configured to rotate in response to a rotating external magnetic field.

14. A system for treating a cardiac valve according to claim 11, the annuloplasty ring further comprising a battery, wherein the motor is an electric motor powered by the battery.

15. A system for treating a cardiac valve according to claim 11, wherein the motor is operative responsive to a stimulus external to the annuloplasty ring.

16. A system for treating a cardiac valve according to claim 11, wherein the annuloplasty ring further comprises a dynamic portion configured to transform in response to a stimulus external to the annuloplasty ring.

17. A system for treating a cardiac valve according to claim 16, wherein the motor drives the dynamic portion.

18. A method for treating a cardiac valve of a patient, the method comprising:
implanting an adjustable annuloplasty ring on or near the cardiac valve annulus of the patient, the adjustable annuloplasty ring comprising a suture coupled to the annuloplasty ring and configured to be anchored to a papillary muscle, wherein the annuloplasty ring selectively adjusts a tension of the suture in response to a stimulus external to the annuloplasty ring;
anchoring the suture to the papillary muscle of the cardiac valve of the patient; and
applying an external stimulus to the annuloplasty ring to adjust the tension of the suture and thereby reposition the papillary muscle relative to the cardiac valve annulus.

19. The method for treating a cardiac valve of claim 18, wherein the external stimulus is applied with the patient postoperatively healed.

20. The method for treating a cardiac valve of claim 18, wherein the adjustable annuloplasty ring comprises a motor, and wherein applying the external stimulus comprises selectively driving the motor in a forward or reverse direction to selectively adjust the tension of the suture.

21. The method for treating a cardiac valve of claim 20, wherein the motor is magnetic, and wherein applying the external stimulus comprises rotating a magnetic field external to the magnetic motor so as to drive the magnetic motor.

22. The method for treating a cardiac valve of claim 18, wherein the external stimulus comprises an electrical impulse.
